(19) United States
(12) Patent Application Publication
(10) Pub. No.: US 2022/0218879 A1
(43) Pub. Date: Jul. 14, 2022

(54) A LUBRICIOUS, THERAPEUTIC AND ABRASION-RESISTANT COATING FOR DEVICES AND METHODS FOR PRODUCING AND USING THEREOF

(71) Applicant: ISRAEL PLASTICS AND RUBBER CENTER LTD., Haifa (IL)

(72) Inventors: Dan LEWITUS, Herzliya (IL); Yael ROTH, Tel Aviv (IL)

(21) Appl. No.: 17/603,433

(22) PCT Filed: Apr. 14, 2020
(86) PCT No.: PCT/IL2020/050437

§ 371 Date: Oct. 13, 2021

(30) Foreign Application Priority Data
Apr. 15, 2019 (IL) 266050

(51) Int. Cl. A61L 29/06; A61L 29/08; A61L 29/16; A61L 33/00; A61L 33/06

(52) U.S. Cl. CPC A61L 29/06; A61L 29/085; A61L 29/16; A61L 33/0041; A61L 2420/02; A61L 33/0082; A61L 2300/42; A61L 2400/18; A61L 33/064

(57) ABSTRACT

There is provided herein a method of coating a polyurethane surface of an insertable medical device, the method comprising obtaining an insertable medical device or a part thereof comprising a polyurethane surface; performing a direct thiolization of the polyurethane surface to produce thiolated polyurethane surface comprising free thiol groups, the direct thiolization comprises a direct reaction between a secondary amine of the polyurethane surface and ethylene sulphide (ES) to form a covalent bond between the amine and the free thiol group; and reacting the thiolated polyurethane surface with a therapeutic/antithrombogenic compound having a vinyl/methacrylate functional group through thiol-ene click reaction, to produce an insertable medical device coated with a therapeutic/antithrombogenic and abrasion (delamination)-resistant coating.

### TECHNICAL FIELD

The present disclosure relates generally to a lubricious, therapeutic/anti-thrombogenic and abrasion-resistant coating for polyurethane insertable medical devices such as, but not limited to, intravascular catheters.

### BACKGROUND

Intravascular devices, such as guidewires and catheters, are crucial to modern-day medical practice. Such medical devices administer parenteral nutrition fluids, drugs, intravenous fluids, and monitor the hemodynamic status of critically ill patients. Thus, the surface interactions with biological systems is of major importance [1], [2].

Catheters can be divided into two broad categories according to the duration of catheterization: 1) temporary—used for short-time vascular access; 2) indwelling—used for long-term [1]. Catheters and medical tubing are commonly made of synthetic materials, including silicones, polyurethanes, polyamides, polyolefins, and polyvinylchloride (PVC). While these materials tend to be mechanically stable and chemically inert, the use of synthetic materials has created several problems [3].

Several perils are associated with the use of intravascular devices.

First, the insertion of the catheter through the mucous membranes or the vascular surfaces of a patient inevitably results in irritation of the area in immediate contact with the device. Soon after the insertion of almost all catheters, a fibrin sheath is formed around the catheter. This fibrin sheath, in case of long term catheterization or poor handling, is the onset of acute thrombosis [4]. Additional damage and an appreciable amount of discomfort to the patient are caused as the result of the high coefficient of friction (COF) between the blood vessel and the catheter surface, as well as during any subsequent movement by the patient. The problem tends to become more acute as storage time (or implantation time) of the device is increased. Although lubricants may be used to minimize initial friction, they are difficult to keep in place and may complicate handling of the devices. Further, the use of lubricants may increase the potential for infection, depends on the interaction between the lubricant and the patient's biological systems [3], [5].

Second, fouling occurs. Synthetic materials are generally not biocompatible or lubricious, especially when directly exposed to bodily fluids, particularly blood. Undesirable physiological reactions such as thrombosis or bacterial infection may result because the synthetic surfaces attract proteins and other physiological fluid (fouling). The presence of micro-cavities or micro-fractures on the surface of an intravascular device allows the bacteria to anchor and provides temporary protection for the microbes from the action of host fluids, allowing the stabilization of their binding [1], [3]. This may result in the onset of local or systemic infections [1], [6], [7].

Of all catheters placed, between 42% and 100% develop fibrin sheaths, and between 20% to 40% develop pericatheter thrombus [4]. Once a pericatheter thrombus or fibrin sheath occurs, the patient is predisposed to infection. Furthermore, pericatheter infection increases the risk of thrombosis [8].

Therefore, a catheter coating that enhances the ease of insertion and evades fouling, thereby decreases the risk of injury to a patient, represents an important advancement in the field of intravascular medical devices.

Water soluble coating materials, such as hydrogels, dissolve or swell in an aqueous environment, are thus capable of manifesting lubricity while in a wet state. These materials are popular because they provide excellent lubricity and biocompatibility. However, they may be sensitive to moisture. Premature moisture absorbance can provide sticky or tacky texture, sometimes lead to delamination of the coating [5].

Additionally, in cases where large numbers of bacteria can attach to the surface of the device early after implantation and create a biofilm, they are shielded from the effects of the antimicrobial agent while encased in their polysaccharide-based biofilm and are free to reproduce. In this situation, bacterial colonies are tough to kill [2].

During the recent decades, there has been a significant growth in the field of research and development of various coatings to overcome the common problems associated with the insertion of intravascular catheterization in general, particularly catheters.

At November 2015, the Food and Drug Administration (FDA) declared that hydrophilic and/or hydrophobic coatings may separate (e.g., peel, delaminate) from medical devices and potentially cause serious injuries to patients. Delamination of coatings can be caused by a variety of factors, including the complexity of the procedure and issues with device design or manufacturing processes [9].

Since Jan. 1, 2010, there have been 11 recalls from various manufacturers associated with these coatings peeling or flaking off of medical devices. In addition, since Jan. 1, 2014, the FDA has received approximately 500 Medical Device Reports (MDRs) describing separation of hydrophilic and/or hydrophobic coatings on medical devices such as guidewires and catheters. Serious injuries associated with the peeling of coatings reported in MDRs included the persistence of coating fragments in patients, requiring surgical intervention to mitigate the consequences, adverse tissue reactions, and thrombosis [9].

Lubricious Coatings

When it comes to function, there are similarities and differences between hydrophobic and hydrophilic coatings. The main parameter for distinguishing between hydrophobic and hydrophilic surfaces is contact angle. Hydrophobic surfaces present a contact angle greater than 90 degrees, and it can be as high as 150 degrees. Hydrophilic surfaces always have contact angles less than 90 degrees and usually less than 50 degrees. Hydrophilic coatings absorb water, and most of them are in fact comprised of more than 90% water when wetted [2], [10].

Although both types of coatings have relatively low coefficients of friction compared with common substrates found in medical devices, hydrophilic coatings tend to be an order of magnitude more lubricious. Some of the best hydrophobic coatings offer coefficients of friction in the range of approximately 0.15 to 0.3. By contrast, hydrophilic coatings that claim to be exceptionally lubricious have coefficients of friction in the range of 0.005 to 0.2 when wetted [10].

In the handling of catheters it is desirable to have them not slippery for handling but protecting the patient by becoming slippery when contacting an aqueous fluid [11], [12]. The hydrophilic character of hydrophilic coatings provides lubrication and lowering the COF between the blood vessels and the surface of the device. Thus, the initial force that is required for the insertion of the catheter is reduced. Furthermore, bacteria are better adsorbed onto hydrophobic surfaces [3], [2].

Hydrophilic lubricious coatings reduce the potential for various infections by significantly reducing protein adherence to the substrate. However, lubrication itself does not ensure the prevention of developing of another phenomenon. For example, central venous catheters (CVCs) and peripherally inserted central catheters (PICCs) have serious potential to cause life-threatening sepsis, and catheter infection rates are 5.3 per 1,000 catheter days [2], [13], [14].

Thus, another current approach in hydrophilic coating technology is to have surfaces with specific chemical species and charges, thus protein adsorption can be delayed, which can directly or indirectly affect attachment of bacteria. Doing this cuts off the process of colonization, and if the numbers of bacteria in the local area can be kept low, biofilm formation can be reduced or delayed [2].

Biological Activity in Hydrophilic Coatings

Approaches of catheter coating today involve the integration of anti-bacterial and anti-thrombogenic within the coating. These coatings can not only provide lubricity and biocompatibility, but they also can serve as a drug reservoir for a local drug delivery [13], [14]. Some hydrophilic coatings employ anticoagulant agents like heparin, which ultimately affects clotting by reducing fibrin formation. However, while the direct administration of heparin or other anticoagulants (e.g. hirudin or citric acid) is effective reducing blood coagulation, it also presents the undesirable risk of uncontrollable patient bleeding [3].

Conventional medical practices aimed at preventing thrombosis include the direct administration of anticoagulant agents such as heparin to patients who are exposed to blood-contacting medical devices and apparatus [3]. However, while the direct administration of heparin or other anticoagulants (e.g. hirudin or citric acid) is effective reducing blood coagulation, it also presents the undesirable risk of uncontrollable patient bleeding [3].

Another driver in the area of antimicrobial coatings is the new Medicare rule requiring hospitals to cover the cost of nosocomial infections arising from catheters. The rule gives incentive to hospitals to create and use more methods to reduce infections [2].

Antimicrobial impregnated catheters have been shown to reduce catheter infection rates. However, as technology for releasing antimicrobial agents from hydrophilic surfaces matures, it becomes evident that other approaches may be equally or more effective. When releasing an antimicrobial agent from a coating, the local concentration of the agent reaches levels toxic to targeted bacteria species, but for devices with long implantation times (>21 days), the release drops off and the local concentration of antimicrobial agent dips below inhibitory levels. For substances such as antibiotics, this can initiate drug resistance if some bacteria are residing in the area [2].

Covalently bonding anti-thrombogenic coatings using prior art techniques often involved relatively harsh conditions and strong chemical solutions or exotic polluting solvents [3].

Hydrophilic coating was disclosed in U.S. Pat. No. 2,768,909, filed by DuPont in 1953. U.S. Pat. No. 2,768,909 described a two-layer system, where a primer coat or a bonding layer is first placed over the substrate. This bonding layer tend to be relatively hydrophobic, thus provides for a consistent binding for a top coat [2], [10], [15].

Since then, hydrophilic coatings have come a long way in the medical field. The market for hydrophilic coatings in medical devices is expanding by 25% annually.

U.S. Pat. No. 4,100,309 filed in 1977 and assigned to Biosearch Medical Products, suggested a hydrophilic lubricious coating comprises a polyvinylpyrollidone (PVP)-polyurethane (PU) interpolymer. The coating was advantageous in that the applying method was dipping. Thus, the thickness of the coating is not limited to a few molecular monolayers as in the case of other methods, such as chemical or radiation grafting, and may be applied in thicknesses of several hundred micrometers. Additionally, the coatings were non-reactive with respect to living tissue and were non-thrombogenic when in contact with blood. However, this method is limited to substrate materials which have good adherence to polyurethanes [11].

An aliphatic non-crossslinked polyurethane-polyethylene oxide (PEO) was disclosed in U.S. Pat. No. 5,041,100 filed in 1989, assigned to Cordis Corporation. Advantages of this coating included the use of aqueous dispersion, restricting the need of inflammable or toxic materials and the ability to add water dispersible therapeutic agents as coating ingredients. However, this coating method was limited to polyurethane and stainless steel substrates [12].

U.S. Pat. No. 6,176,849, filed in 1999 and assigned to Scimed Life Systems, attempted to overcome the problems of premature moisture uptake in hydrogel. A first hydrogel layer provides an improved lubricity and a second hydrophobic top coat prevents the prematurely moisture absorption by the hydrogel coating. The hydrophobic top coating comprises a hydrophilic surfactant which acts as a carrier to facilitate removal of the hydrophobic top coating upon coming in contact with an aqueous environment, such as body fluids, particularly blood. The main risk associated with these coatings is the release of hydrophobic particles into the blood stream. These foreign particles can flow through the bloodstream and reach undesirable physiologic systems and disrupt their proper functioning [5].

In U.S. Pat. No. 6,340,465 filed in 1999, assigned to Edwards Lifescience Corporation, improved hydrophilic coatings have been disclosed, using water-based polymer formulations (soluble or dispersed). These coatings are comprised of coupling agents and polyfunctional polymers which are able to form a crosslinked coating and are capable of entrapping or affixing hydrophilic and/or lubricious compounds, as well as antithrombogenic or anticoagulant agents [3].

Later on, U.S. Pat. No. 8,513,320, filed in 2008, assigned to DSM IP Assets B.V., disclosed a method for providing a durable hydrophilic coating by applying PUR primer on PEBAX surface, and PEG hydrophilic coating that was UV cured as a top coat [16].

U.S. Pat. No. 9,244,195 filed in 2012, a method was disclosed for making a silicone hydrogel contact lens having a nano-textured surface which mimics the surface texture of the cornea of human eye. Swelling a silicone hydrogel contact lens in a solution containing a polyacrylic acid (PAA) polymeric primer coating which is dissolved in an organic solvent. The lens is swelled once in contact with the organic solvent, allowing the PAA molecules to penetrate under the lens surface. Another solvent provides the reshrinking of the lens and provide a mechanical interlocking of the primer coating. A water soluble, crosslinkable hydrophilic top coat consists of poly(acrylamide-co-acrylic acid) covalently bonded to the primer coating through additional functional groups [17].

Zwitterions as Natural Antifouling Agents

Since the late 1970s much attention has been devoted to the use of lipid-like materials for modifying surfaces to improve their compatibility with biological systems. Although these are common materials used in the field of hydrophilic lubricious coatings, there is a growing ambition to try to mimic materials that are commonly present in the human body [18].

In 1977, Zwaal et al. demonstrated that the bilayer of phospholipids around the cell is asymmetric. While the inner cytoplasmic surface consists of a larger proportion of negatively charged phospholipids, which are known to be thrombogenic, the main lipid components that constitute the outside surface are known to be zwitterions, particularly phosphatidylcholines [19].

Phosphatidylcholine are a class of phospholipids that incorporate choline as a head group.

Choline Structure [20]:

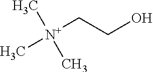

They are a major component of biological membranes and can be easily extracted from available sources, such as egg yolk or soybeans, using hexane.

Common hydrophilic coatings today consist of polyvinylpyrolidone (PVP), polyethylene glycol (PEG), polyurethanes, polyacrylic acid (PAA), polyethylene oxide (PEO), and polysaccharides [2]. While hydrophilic and neutral polymers such as polyethylene glycol (PEG) can form a hydration layer via hydrogen bonds, zwitterions form a hydration layer via electrostatic interactions. Zwitterions are capable of binding a significant amount of water molecules and therefore are potentially excellent candidates for super-low fouling materials [21].

Zwitterions are characterized by possessing an equal number of both positively and negatively charged groups within a molecule thus maintaining overall electrical neutrality and was shown to be non-thrombogenic [21], [19]. Polyzwitterionic materials can be further classified into polybetaines, such as 2-methacryloyloxylethyl phosphorylcholine (MPC), sulfobetaine methacrylate (SBMA) and carboxybetaine methacrylate [22].

Phosphorylcholine

Phosphorylcholine is a polar head group of some phospholipids that are members in the family of phosphatidylcholine.

Phosphorylcholine Headgroup Structure [23]:

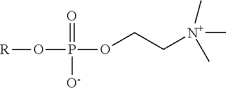

MPC is a polybetain containing phosphorticholine head group and was widely studied for its antifouling and antithrombogenic capabilities.

2-ethacryloyloxylethyl phosphorylcholine (MPC) Structure:

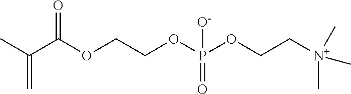 [SMILES: C=C(C)C(=O)OCCOP(=O)([O-])OCC[N+](C)(C)C]

In 2003, the research group of Professor Lloyd [24], designed PC-based polymers that have been used in a variety of medical device applications to improve biocompatibility. They showed that the presence of 2-methacryloyloxyethyl phosphorylcholine-co-lauryl methacrylate (MPC-co-LMA2) inhibits protein adsorption.

2-Methacryloyloxyethyl-phosphorylcholine (MPC) polymer belong to the family of phosphatidylcholines. The MPC structure is composed of a methacrylate and PC head group, and the side chain consists of a phosphate anion and a quaternary ammonium cation.

The high water retention properties, anti-fouling and non-toxic nature of MPC polymers have made them a widely used material for biomedical applications. For example, MPC scaffolds have been used extensively for tissue engineering applications [21], [25].

MPC-based polymers have been shown to significantly reduce protein adsorption compared to relevant controls and have been widely used for various applications [18]. Antifouling and antithrombogenic coatings have been developed based on PC functioning [26], [27], [28].

In 2005, S. Chef et al. [21] synthesized a phosphorylcholine (PC)-thiol for the evaluation of protein absorbance by PC self-assembled molayer (SAM). They demonstrated that zwitterionic PC SAMs are highly resistant to protein adsorption, when balanced charge and minimized dipole are two key factors for their nonfouling behavior. PC SAMs have very low protein adsorption when the N/P ratio is close to 1:1 and the charges are balanced. PC head groups have similar packing densities to membrane lipids and prefer to have an antiparallel orientation for dipole minimization.

Tiolated Phosphorylcholine:

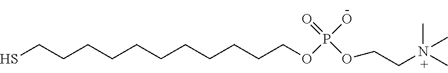

Polyethyleneimine (PEI)

PEI is a water soluble, highly reactive cationic polymer which is made by a ring opening polymerization of ethyleneimine. In its common structure, PEI is partially brunched polymer containing primary, secondary and tertiary amines. PEI had been widely explored for its gene delivery potential. Thanks to its high cationic density, PEI retains a substantial buffering capacity at virtually any PH. The use of PEI had been extended in the last decades to serve as an anchor agent for coatings.

Synthesis of PEI:

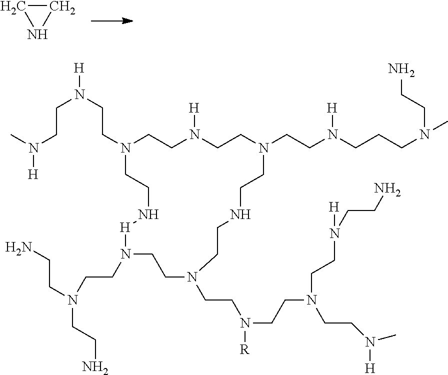

In 2002, crosslinked PEI was presented by Edwards Lifescience Corporation [3] as an anchor agent for the purpose of coating of polyester and polyethylene surfaces. They demonstrated that the coatings are lubricious and capable of being antimicrobial, protein repelling and antithrombogenic; antithrombogenic agents, such as heparin, can be entrapped or affixed to the coating.

Thiol functional groups have been introduced onto particle surfaces to covalently conjugate drugs or targeting groups. Thiolated PEI was made by stirring of low molecular weight PEI (LMPEI) with 2-methylthiirane in ethanol in order to further produce disulfide crosslink. Another thiolated PEI was reported in the literature in 2013 [29], when thiolization of PEI occurred through two main steps, including the substitution of disulfide-containing pendant chain onto free amines of the PEI; cleaving of disulfide linkage.

Disclosed in ES 262,821,0T3 in 2015, PEI covalently bonded anti-thrombogenic coating was disclosed by Regensburg University [30]. The coating comprises anti-thrombogenic material, which is covalently bound to a polyurethane surface through PEI as a third-party agent. An amide bond is formed between the surface of a polyurethane surface and PEI. An additional covalent bond is formed between the PEI and the anti-thrombogenic substance. Applying this coating required surface activation of the polyurethane by CO₂ or air plasma. In this research, the lubricity of the coating has not been explored.

There is still a need in the art for coatings that demonstrate antithrombogenicity with high lubricity and are resistance to delamination.

### SUMMARY

Aspects of the disclosure, according to some embodiments thereof, relate to a new lubricious, antimicrobial, antithrombogenic and durable coating, which may be applied to insertable medical devices such as, but not limited to, intravascular devices (IVD), such as catheters, stents and guidewires and/or any other device configured for intra cavity insertion, temporary, indwelling or implantable. More specifically, but not exclusively, aspects of the disclosure, according to some embodiments thereof, relate to a lubricious, antimicrobial, antithrombogenic ‘stock product’ synthesized as a preliminary step to the application of the coating on the substrate. The term “insertable medical device(s)” may refer to any medical device that is configured to or has a part that is configured for insertion and/or implantation in the human body.

According to some embodiments, the lubricious, antimicrobial, antithrombogenic ‘stock product’ for coating includes a therapeutic compound, such as antimicrobial compound, and/or antithrombogenic compound, that has a vinyl functional group (such as for example but not limited to (2-methacryloyloxylethyl phosphorylcholine (MPC) known as an antithrombogenic agent), covalently attachable to the insertable medical device (e.g., IVD) surface through a third-party agent, such as, thiolated polyethyleneimine (PEI-SH), for example, thiolated-brunched polyethyleneimine (bPEI-SH).

Advantageously, the PEI-SH, such as the bPEI-SH not only serves as the linker between the PC and the surface of the insertable medical device (e.g., IVD), but is also the main source for the lubricity of the coating. According to some embodiments, branched PEI enables to increase the binding sites for vinyl groups/methacrylate groups of therapeutic compounds, such as MPC molecules, thus increases the therapeutic/antithrombogenic properties of the coating.

According to some embodiments, the stock product includes polyethyleneimine-thiol-zwitterionic methacrylate (PEI-S-ZWIMA), which is composed of zwitterionic methacrylate (ZWIMA) covalently bound to thiolated polyethyleneimine (PEI-SH). According to some embodiments, the stock product includes polyethyleneimine-thiol-2-methacryloyloxylethyl phosphorylcholine (PEI-S-MPC), which is composed of 2-methacryloyloxylethyl phosphorylcholine (MPC) covalently bound to thiolated polyethyleneimine (PEI-SH).

According to some embodiments, the stock product includes polyethyleneimine-thiol-2-methacryloyloxylethyl phosphorylcholine (PEI-S-MPC), which is composed of 2-methacryloyloxylethyl phosphorylcholine (MPC) covalently bound to polyethyleneimine (PEI) via ethylene sulphide (ES) as an anchoring group between PEI and MPC.

According to some embodiments, the coating material in the stock product consists essentially of PEI-S-MPC. According to some embodiments, the stock product is devoid of coating materials other than PEI-S-MPC. According to some embodiments, the stock product may include only residual amounts of coating materials other than PEI-S-MPC.

According to some embodiments, the stock product, namely, PEI-S-MPC for coating a medical device may be produced as follows:
obtaining a thiolated polyethyleneimine (PEI-SH), for example, thiolated-brunched polyethyleneimine (bPEI-SH), and
reacting the thiolated polyethyleneimine (PEI-SH) with 2-methacryloyloxylethyl phosphorylcholine (MPC) through thiol-ene click reaction to produce polyethyleneimine-thiol-2-methacryloyloxylethyl phosphorylcholine (PEI-S-MPC).

According to some embodiments, the thiolated polyethyleneimine (PEI-SH) may be synthesized through ring opening of ethylene sulphide (ES). Ethylene sulfide (ES) is highly reactive, due to the natural cyclic stress of three membered ring located 60° from each other performing a triangle. The thiolation reaction occurs between both the primary and secondary amines groups and the unstable monomer of ES.

An example of a scheme for the preparation of a ‘stock product’, such as, PEI-S-MPC is shown in FIG. 1, in accordance with some embodiments. bPEI contain 25% primary, 50% secondary and 25% tertiary amine groups.

According to some embodiments, the stock product is applied on the substrate, such as an insertable medical device (e.g., IVD), via the following steps:

1. PU surface is functionalized using diisocyanate substance. One isocyanate group is covalently attached to the surface via free amines, while the other isocyanate group is free and available for further reaction.

2. Second, the covalent attachment of the stock product to the isocyanate free groups takes place via free primary and secondary amines which present in the stock product, resulting in a urea bond.

Advantageously, in accordance with some embodiments, the coating stock product can be produced in advance and optionally in a manufacturing location/facility different from the location of the actual application of the coating onto the medical device.

Advantageously, in accordance with some embodiments, the application of the coating material (such as, but not limited to PEI-S-MPC) onto the substrate (e.g., functionalized PU of the medical device) may then be performed in a straight forward dip-coating technique, allowing high throughput and scalability of the process. The resulting product is a coated substrate with covalently attached lubricious, abrasion (delamination)-resistant, antimicrobial and antithrombogenic coating.

According to an aspect of some embodiments, there is provided a method for preparing a stock product for use as a coating material for coating a polyurethane surface of an insertable medical device, the method comprising: obtaining a thiolated polyethyleneimine (PEI-SH); and reacting the thiolated polyethyleneimine (PEI-SH) with a therapeutic/antithrombogenic compound having a vinyl/methacrylate functional group through thiol-ene click reaction to produce a stock product comprising polyethyleneimine-thiol-therapeutic/antithrombogenic compound conjugate having free primary and/or secondary amines capable of binding to an activated surface of the insertable medical device.

The therapeutic/antithrombogenic compound having a vinyl/methacrylate functional groups may include zwitterionic methacrylate. The zwitterionic methacrylate may include sulfobetaine methacrylate, phosphorylcholine methacrylate or a combination thereof. According to some embodiments, the zwitterionic methacrylate may include 2-methacryloyloxylethyl phosphorylcholine (MPC) and the stock product may include polyethyleneimine-thiol-MPC (PEI-S-MPC) conjugate.

According to some embodiments, the therapeutic/antithrombogenic compound having a vinyl/methacrylate functional groups may include Linalool, Limonene, Citral or any combination thereof.

According to some embodiments, obtaining the thiolated-polyethyleneimine (PEI-SH) may include reacting polyethyleneimine (PEI) with ethylene sulphide (ES), halogen-alkyi thiol, cysteine, bromopyridine thiol, bromobenzoxazole thiol, chloropyridine thiol, halobenzo thiazole thiol, chloropyrimidine thiol, halo-phenyl thiazole thiol or any combination thereof.

According to some embodiments, the polyethyleneimine (PEI) and/or the thiolated polyethyleneimine (PEI-SH) may include brunched polyethyleneimine (bPEI) and/or thiolated-brunched polyethyleneimine (bPEI-SH), respectively.

According to an aspect of some embodiments, there is provided a method for preparing polyethyleneimine-thiol-2-methacryloyloxylethyl phosphorylcholine (PEI-S-MPC) for use as a stock product for use as a coating material for coating a surface of an insertable medical device, the method comprising: obtaining a thiolated polyethyleneimine (PEI-SH); and reacting the thiolated polyethyleneimine (PEI-SH) with 2-methacryloyloxylethyl phosphorylcholine (MPC) through thiol-ene click reaction to produce brunched polyethyleneimine-thiol-2-methacryloyloxylethyl phosphorylcholine (PEI-S-MPC) having free primary and/or secondary amines capable of binding to an activated surface of the insertable medical device. Advantageously, the pre-synthesis of the PEI-S-MPC complex, allows for a “grafting-to” process onto the activated polyurethane surface, in a single step, making the process industrially valid.

According to some embodiments, obtaining the thiolated-polyethyleneimine (PEI-SH) may include reacting polyethyleneimine (PEI) with ethylene sulphide (ES), halogen-alkyi thiol, cysteine, bromopyridine thiol, bromobenzoxazole thiol, chloropyridine thiol, halobenzo thiazole thiol, chloropyrimidine thiol, halo-phenyl thiazole thiol or any combination thereof.

According to some embodiments, the polyethyleneimine (PEI) and/or the thiolated polyethyleneimine (PEI-SH) may include brunched polyethyleneimine (bPEI) and/or thiolated-brunched polyethyleneimine (bPEI-SH), respectively.

According to an aspect of some embodiments, there is provided a stock product, prepared according to any of the methods disclosed herein, for use in coating an activated polyurethane surface of an insertable medical device.

According to an aspect of some embodiments, there is provided a method of coating a polyurethane surface of an insertable medical device, the method comprising: obtaining an insertable medical device or a part thereof comprising a functionalized polyurethane surface having free isocyanate groups; reacting the functionalized polyurethane surface with a stock product comprising a conjugate of polyethyleneimine-thiol-therapeutic/antithrombogenic compound having free primary and/or secondary amines capable of binding to the free isocyanate groups of the polyurethane surface. The polyurethane surface may be functionalized using diisocyanate substance to produce. The diisocyanate substance may include hexamethylene diisocyanate (HDI), L-lysine diicosyanate (lysine-D), isophorone diisocyanate, phenylene diisocyanate, xylylene diisocyanate, cyclohexylene diisocyanate, alkyl diisocyanate or any combination thereof.

According to some embodiments, the polyurethane functionalizing may include covalently attaching to the polyurethane surface one isocyanate group of the diisocyanate substance while the other isocyanate group of the diisocyanate substance is free and available for further reacting with the free primary and/or secondary amines of the stock product.

According to some embodiments, the method of coating a polyurethane surface of an insertable medical device, may include: obtaining an insertable medical device or a part thereof comprising a functionalized polyurethane surface having free isocyanate groups; reacting the functionalized polyurethane surface with a stock product comprising a conjugate of polyethyleneimine-thiol-2-methacryloyloxylethyl phosphorylcholine (PEI-S-MPC) to produce coated polyurethane (functionalized PU-PEI-S-MPC). Reacting the functionalized polyurethane surface with stock product may be conducted utilizing dip coating technique.

According to some embodiments, there is provided herein another approach, a direct approach, for coating an insertable medical device to form a lubricious, abrasion (delamination)-resistant, antimicrobial and antithrombogenic coating:

Direct Thiolation of Polyurethane using Ethylene Sulfide (ES)

There is provided herein, in accordance with additional or alternative embodiments, a method for the conjugation of a therapeutic/antithrombogenic compound (such as MPC) to PU surfaces. This approach may be used for direct conjugation via thiol-based reactions, such as thiol-ene with allyl or methacrylate bearing molecules (such as MPC), isocyanate bearing molecules, epoxides and the like.

According to an aspect of some embodiments, there is provided a method of coating a polyurethane surface of an insertable medical device, the method comprising: obtaining an insertable medical device or a part thereof comprising a polyurethane surface; performing a direct thiolization of the polyurethane surface to produce thiolated polyurethane surface comprising free thiol groups, the direct thiolization comprises a direct reaction between a secondary amine of the polyurethane surface and ethylene sulphide (ES) to form a covalent bond between an amine and a free thiol group; reacting the thiolated polyurethane surface with a therapeutic/antithrombogenic compound having a vinyl/methacrylate functional group through thiol-ene click reaction, to produce an insertable medical device coated with a therapeutic/antithrombogenic and abrasion (delamination)-resistant coating.

According to some embodiments, the direct thiolization of the polyurethane surface to produce thiolated polyurethane surface includes ring opening of the ES and formation of a specific covalent bond between the thiol (originated from the ES) and the secondary amine (originated from the polyurethane surface). According to some embodiments, and without limitation to a mechanism of action, the secondary amine acts as a nucleophile, and attacks the thiirene (ES) causing ring opening, resulting in thiol formation.

According to some embodiments, the direct thiolization of the polyurethane surface to produce thiolated polyurethane surface is performed without (devoid of) a pre-treatment of the polyurethane surface. Such devoid (avoided) pre-treatment may include for example, plasma treatment of any sort (such as O₂, CO₂ or any other gas plasma), chemical etching, flame pre-treatment, corona pre-treatment and/or any other surface pre-treatment or combination of treatments to the polyurethane surface.

The therapeutic/antithrombogenic compound having a vinyl/methacrylate functional group may include zwitterionic methacrylate. The zwitterionic methacrylate may include, for example, sulfobetaine methacrylate, phosphorylcholine methacrylate or a combination thereof. The zwitterionic methacrylate may include 2-methacryloyloxylethyl phosphorylcholine (MPC) and wherein the coated polyurethane surface is (PU-S-MPC).

According to some embodiments, the therapeutic/antithrombogenic compound having a vinyl/methacrylate functional group may include, for example, Linalool, Limonene, Citral or any combination thereof.

According to an aspect of some embodiments, there is provided an insertable medical device having a polyurethane surface coated according to any of the methods disclosed herein.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1 shows an example of a scheme for the preparation of PEI-S-MPC, in accordance with some embodiments;

FIG. 2 shows an ATR-FTIR spectroscopy of thiolated bPEI and untreated bPEI, according to some embodiments;

FIG. 3 shows a UV-VIS analysis of bPEI-SH using Ellman's reagent, according to some embodiments;

FIG. 4 schematically depicts a mechanism of Ellman's reagent for the detection of thiol groups, according to some embodiments;

FIG. 5 shows a calibration curve for the quantification of free thiol groups using cysteine, according to some embodiments;

FIG. 6 shows the IR spectroscopy of the end product bPEI-S-MPC (prepared according the scheme of FIG. 1), according to some embodiments;

FIG. 7 shows two vials, the vial on the right contains the end ‘stock product’ (bPEI-S-MPC), which is soluble in water, whereas the vial on the left contains bPEI-SH, which is insoluble in water, according to some embodiments;

FIG. 8 shows a reaction scheme between Urethane and isocyanate functional group, according to some embodiments;

FIG. 9 shows a reaction scheme between PU and TSC, according to some embodiments;

FIG. 10 shows an ATR-FTIR spectrum of PU-TSC compared to untreated PU surface and TSC reagent, according to some embodiments;

FIG. 11 shows a scheme of the peak areas that was analyzed for the optimization of the reaction of PU with isocyanate functional group, according to some embodiments;

FIG. 12 shows the influence of the duration (left graph) and the temperature (right graph) of the reaction on the quantity of conjugated molecules according to the integration ratio factor A1157/A1527, according to some embodiments;

FIG. 13 shows a scheme of an application of the ‘stock product’ (bPEI-S-MPC) on PU surface through functionalization of the PU surface, according to some embodiments;

FIG. 14 shows a scheme of a functionalization of PU surface using hexamethylene diisocyanate (HDI) producing PU-HDI, according to some embodiments;

FIG. 15 shows a scheme of a functionalization of PU surface using L-lysine diicosyanate (lysine-D) producing PU-lysine-D, according to some embodiments;

FIG. 16 shows an ATR-FTIR spectrum of PU-HDI (produced in a process according to FIG. 14), according to some embodiments;

FIG. 17 shows an ATR-FTIR spectrum of PU-lysine-D (produced in a process according to FIG. 15), according to some embodiments;

FIG. 18 shows a scheme of an application of bPEI on PU surface through functionalization with HDI, according to some embodiments;

FIG. 19 shows a scheme of the sample series that was made for the characterization of coated PU surfaces, according to some embodiments;

FIG. 20 shows blood-agar petri dishes containing the sample series shown in FIG. 19 and a control dish, that went through JIS Z2801:200 test for antimicrobial properties, according to some embodiments;

FIG. 21 shows COF of the sample series shown in FIG. 19, on PMMA in the presence of PBS, according to some embodiments;

FIG. 22 shows the hemolysis ratio of PU-HDI-bPEI-S-MPC, compared to an uncoated PU surface and a commercial hydrogel coat, which can be found in medical use today, according to some embodiments;

FIG. 23 shows a scheme of direct thiolation of PU surface, according to some embodiments;

FIG. 24 shows a UV-VIS absorbance curve of Ellman's reagent decomposition products after the exposure to thiolated PU surface (PU-SH), according to some embodiments; and

FIG. 25 shows fluorescent microscopy of fluorescein-O-methacrylate grafted PU-SH (A) compared to a reference (B), according to some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation.

Materials and Methods

All chemicals were purchased and used without further purification. Polyethyleneimine (PEI branched, Mw 800 by LS), ethylene sulfide (ES, 98%), 2-methacryloyloxyethyl phosphorylcholine (MPC 97%, ≤100 ppm MEHQ as inhibitor), 2,2-dimethoxy-2-phenylacetophenone (DMPA 99%), Fluorescein-O-methacrylate (95%), hexamethylene diisocyanate (HDI≥99% by GC), dibutyltin dilaurate (DBTDL, 95%) were all purchased from Sigma-Aldrich, IL. 5,5′-dithiobis(2-nitrobenzoic acid) (DTNB, 97%), N-acetyl-L-cysteine (C₅H₉NO₃S, 98+%), L-lysine ethyl ester diisocyanate (97%) were all purchased from Alfa Aesar. Toluene AR-b, ethanol absolute (dehydrated) AR-b, methanol AR-b, diethylether AR were all purchased from Bio-Lab. Biomedical grade of PU was generously donated by Lubrizol.

Synthesis of the Lubricious Antimicrobial Coating Complex (Stock Product)

bPEI-SH was synthesized through ring opening of ES. 6 gr of bPEI were dissolved in a mixture of toluene; ethanol solution (9:1 ratio, respectively) in a 100 ml round flask. The solution was refluxed under nitrogen atmosphere for 15 min, then 400 μl of ES were added dropwise over 1 min. The reaction was refluxed for another 2 hr, following the removal of the solvent mixture by evaporation under reduced pressure. The thiolation of bPEI was analyzed using IR spectroscopy, UV-VIS spectroscopy and fluorescent microscopy.

bPEI-S-MPC was synthesized through thiol-ene click reaction. 800 mg of MPC and 20 mg of DMPA were dissolved in 4.5 ml of methanol and were added to 2 g of bPEI-SH. The mixture was radiated under UV light (20-watt, 365 nm, Analytik Jena US) for 40 min. The solvent was removed by rotary evaporator under reduced pressure. The product was analyzed using IR spectroscopy and elemental analysis.

IR Spectroscopy

Absorption spectrum was obtained using Fourier-transform infrared spectroscopy spectrometer (Bruker, Germany), with attenuated total reflection method (ATR-FTIR). Using OPUS software, 100 scan signals were provided for each sample and the average resolution of the measurement was adjusted to 2 cm⁻¹.

UV-VIS Spectroscopy

DTNB, also called Ellman's reagent, can be used for the detection of free thiol groups using UV-VIS spectroscopy. DTNB reacts with a free sulfhydryl groups to yield a disulfide molecule and 2-nitro-5-thiobezoic acid (TNB). Elevated absorption in the range of 412 nm is associated with the presence of TNB, which indicates for the presence of thiol free groups in a tested sample.

The absorbance was detected using a UV-VIS spectrometer (UV-1650PC, Shimadzu Corporation, Japan). 4.6 mg/ml of bPEI-SH were dissolved in distilled water for the measurement. As a reference, 4.6 mg/ml of bPEI were dissolved in distilled water.

Ellman's reagent protocol enabled the quantification of thiols, based on molar absorptivity of a standard concentration of thiols using cysteine.

Preparation of PU Surfaces

The samples to be coated were prepared by solvent casting onto glass petri dishes. PU resins were dissolved in THF in a concentration of 2% w/t. Air plasma was applied on glass petri dishes (90 mm in diameter) for 5 min, following by casting of 15 ml of 2% w/t PU solution (in THF). After ambient evaporation of the solvent, the casted dishes were dried overnight at 55° C. and held under vacuum.

Coating Application on PU Surfaces

PU surfaces were functionalized using diisocyanate substances. 5% v/v of HDI and 0.25% v/v of DBTDL were added to 15 ml of toluene and the solution was added over a PU solvent casted petri dish. The reaction was conducted for 60 min at 70° C. under orbital spinning of 50 rpm.

The application of the coating complex onto functionalized surfaces was conducted in the same reaction procedure. 1 gr of bPEI/bPEI-SH/bPEI-S-MPC and 0.25% v/v of DBTDL were added to toluene and the solution was spread over a functionalized surface. The reaction was conducted for 60 min at 70° C. under orbital spinning of 50 rpm.

The coated surfaces were analyzed using elemental analysis. The coefficient of friction (COF) of coated PU was measured in PBS. Fibrinogen absorption assay and antimicrobial tests were performed on coated PU surfaces.

X-Ray Photoelectron Spectroscopy (XPS)

The X-ray Photoelectron Spectroscopy (XPS) measurements were performed on a Kratos Axis Ultra X-ray photoelectron spectrometer (Karatos Analytical Ltd., Manchester, UK). High resolution XPS spectra were acquired with monochromatic Al Ka X-ray radiation source (1,486.6 eV) with 90° takeoff angle (normal to the analyzer). The pressure in the chamber was 2·10-9 Torr. The high-resolution XPS spectra were collected for with pass energy 20 eV and step 0.1 eV. Data analyses were performed using Casa XPS (Casa Software Ltd.) and Vision data processing program (Kratos Analytical Ltd.).

Elemental Analysis

Determination of atomic percent of the coating complex was performed using elemental analysis. C, N, H and O percentage was measured using the Thermo Flash 2000 CHN-O Elemental Analyzer. This system uses a simultaneous flash combustion method (950-1060° C.) for CHN and pyrolysis of oxygen to convert the sample elements to simple gases. The gases are detected as a function of their thermal conductivity. The determination of S, P percentage is done using the Anton Paar Microwave Induced Oxigen Combustion (MIC) for the decomposition of organic samples and by Ion chromatography analysis using a Dionex IC system.

Coefficient of Friction (COF) Measurements

The COF of coated surfaces had been measured in PBS. For this purpose, a bath was constructed from PMMA to fit the standard apparatus to perform a standard COF test according to ASTM 1894. The bath was filled with 30 ml of PBS and each sample was tested for 25 cycles. After 10 and 20 cycles, 1.5 ml of the test liquid were collected to further evaluation of particulates. After 25 cycles, the remained PBS had been collected to an empty vial. The tested surfaces were analyzed using scanning electron microscopy (SEM). The PBS was analyzed using particle size analyzer to identify and measure particles and was seen under tunneling electron microscopy (TEM) to check for amorphous particles.

To evaluate the influence of the coating on the COF values of the surfaces, a standard measurement had been conducted, according to some adjustments

Antimicrobial Test

The antimicrobial activity of the coating was evaluated using the JIS Z2801:2000 test. The tested samples (5 mm in diameter) were incubated with E. coli bacteria for 24 hours at 37° C. in a humid atmosphere. Then, the samples were sonicated to detach all bacteria on the surface. The sonicated liquid was cultured on blood-agar petri dishes, following by another overnight incubation at 37° C. in a humid atmosphere. The antimicrobial activity of each sample was determined by the number of colony forming units developed over the culture petri dishes.

Hemolysis Test

The degree of hemolysis was evaluated as follows: each sample (5 mm in diameter) was soaked in 160 μl of PBS at 37° C. for 30 min. 510 μl of fresh blood from healthy pigs (containing 6% v/v of 20 mg/ml of potassium oxalate) were added to each sample and the samples were incubated at 37° C. for another 60 min. then, the samples were centrifuged at 1350 rpm for 5 min. the absorbance of the supernatant solution was measured using a plate reader at a wavelength of 545 nm. The hemolytic ratio (HR) was calculated by the following equation: HR(%)=(Aₛ−A_{nc})/(A_{pc}−A_{nc}), were Aₛ is the obtained absorbance of the tested sample, A_{nc} and A_{pc} are the absorbance of the negative control (0.02% v/v of diluted blood in PBS) and positive control (0.02% v/v of diluted blood in DI water), respectively.

### EXAMPLES

Synthesis of bPEI-SH

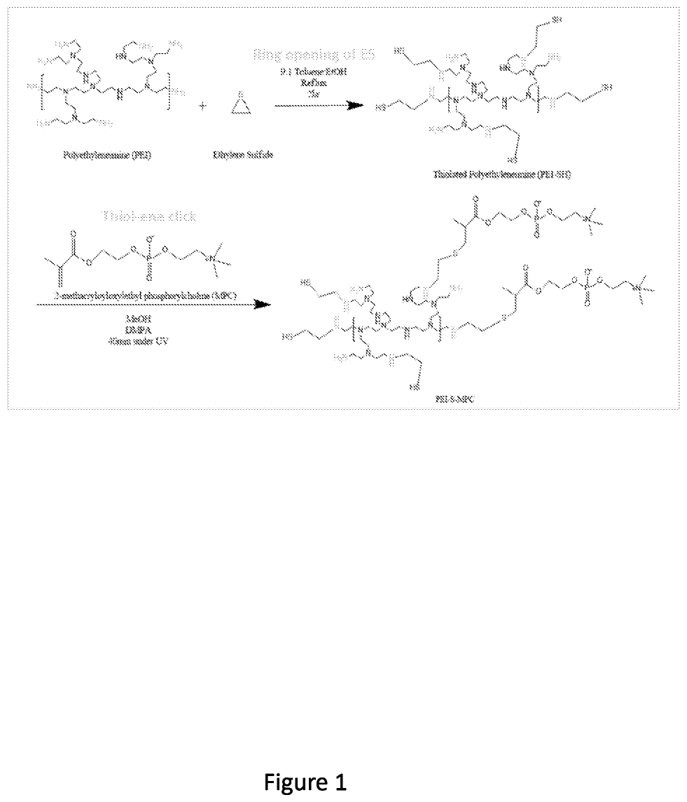

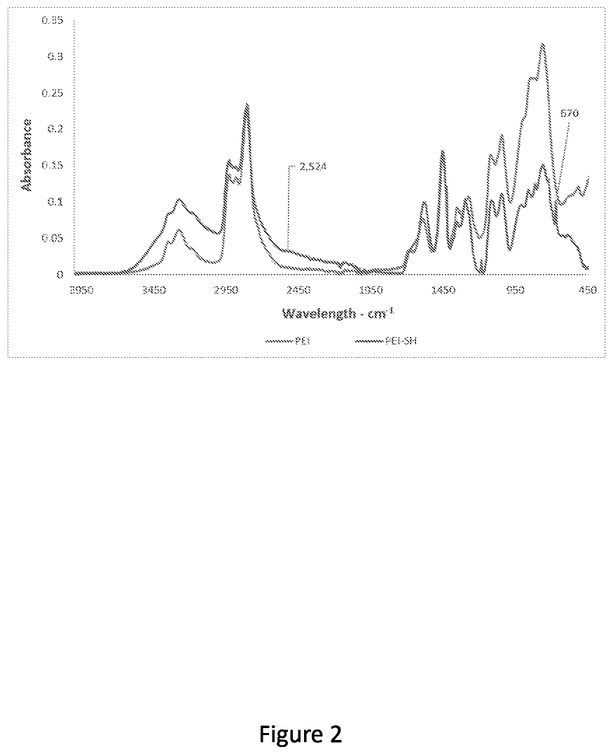

Reference is now made to FIG. 1, which shows an ATR-FTIR spectroscopy of thiolated bPEI and untreated bPEI, according to some embodiments.

Both peaks at 670 cm−1 and 2523 cm−1 are known for common frequencies of thiol stretch. This could be an indication for the presence of thiols in the bPEI. However, detecting thiols in IR spectroscopy can be misleading. Although thiols can be considered as analogs of the equivalent oxygenated compounds, C—S—H and C—S stretching vibrations give rise to weak absorptions in the IR spectrum. Thus, UV-VIS analysis was performed to support the IR findings.

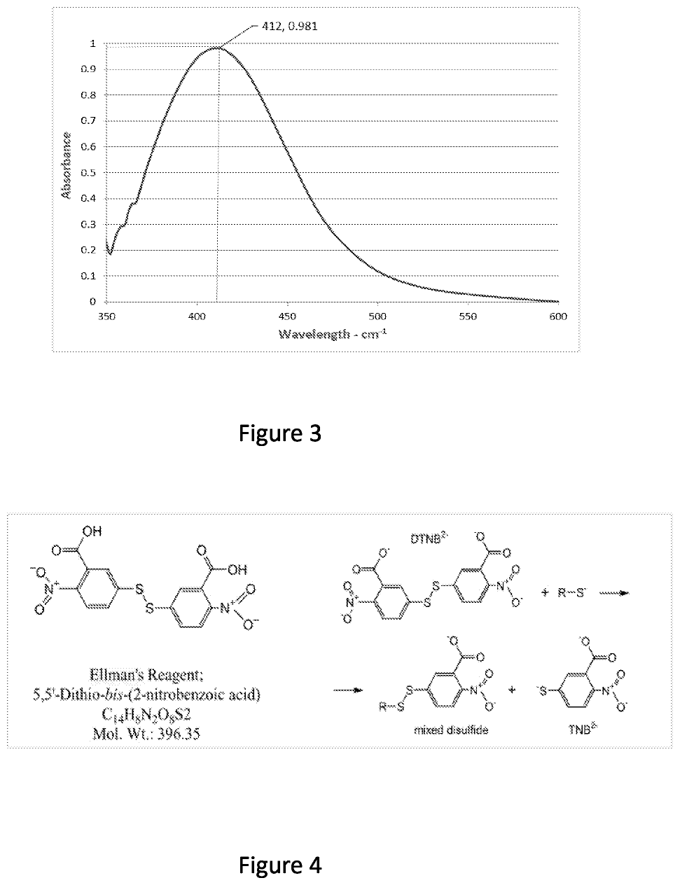

Reference is now made to FIG. 3, which shows a UV-VIS analysis of bPEI-SH using Ellman's reagent, according to some embodiments and to FIG. 4, which schematically depicts a mechanism of Ellman's reagent for the detection of thiol groups, according to some embodiments.

Following a standard protocol for using the Ellman's reagent, thiol concertation in an unknown sample can be expressed by Equation 1, when c is the concentration of thiols in the sample, A is the absorbance at 412 nm, b is the size of the spectrophotometric cuvette in cm and E is the molar absorptivity at 412 nm.

$$Equation 1. The concentration of thiol free groups$$

$$C = \frac{A}{bE}$$

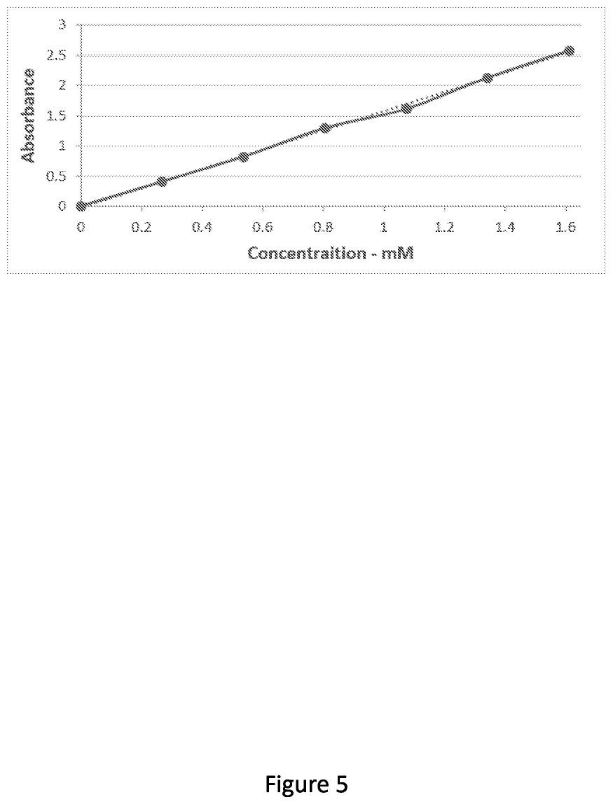

Reference is now made to FIG. 5, which shows a calibration curve for the quantification of free thiol groups using cysteine. The accuracy was found to be R²=0.9982 and the equation is y=1.5824x−0.0135

Thiol-ene Click for the Conjugation of MPC

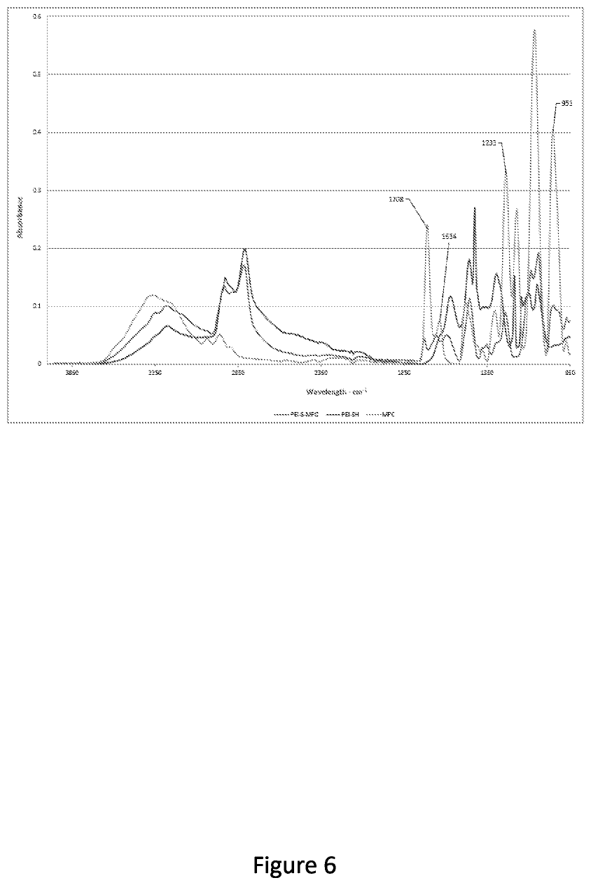

Reference is now made to FIG. 6, which shows the IR spectroscopy of the end product bPEI-S-MPC (prepared according the scheme of FIG. 1), according to some embodiments. The peaks at 1708 cm⁻¹, 1634 cm⁻¹ and 951 cm⁻¹ correspond to carbonyl functional group, alkene double bond and (N⁺(CH₃)₃) group, respectively, which are present in MPC molecule. The peak at 1233 cm⁻¹ correspond to the phosphonate group. All of these peaks could be found on the end product bPEI-S-MPC as well.

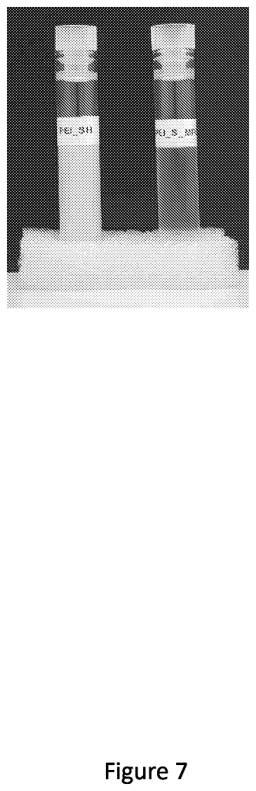

Table 1 shows the atomic percent distribution that was obtained from elemental analysis. After the thilation of bPEI, 3.27% of the detected atoms was found to be sulfur. Phosphorous was detected only in the end ‘stock product’ and its atomic percentage was 3.03%. Visually, there was no significant change between bPEI-SH and bPEI-S-MPC. However, the end ‘stock product’ (bPEI-S-MPC) is soluble in water, where bPEI-SH is insoluble in water, as could be seen in FIG. 7.

TABLE 1
Elemental analysis results of the ‘stock product’
(prepared according the scheme of FIG. 1):

| | bPEI | bPEI-SH | bPEI-S-MPC |
|---|---|---|---|
| C % | 52.37 | 49.67 | 47.91 |
| H % | 10.49 | 10.64 | 9.19 |
| N % | 34.36 | 30.46 | 23.06 |
| S % | 0 | 3.27 | 2.17 |
| O % | 2.78 | 5.24 | 15.28 |
| P % | 0 | 0 | 3.03 |

As disclosed herein, in accordance with some embodiments, the stock product may be prepared as a preliminary step for the coating, thus significantly simplifies the coating application itself.

Functionalization of the Surface of Polyurethane

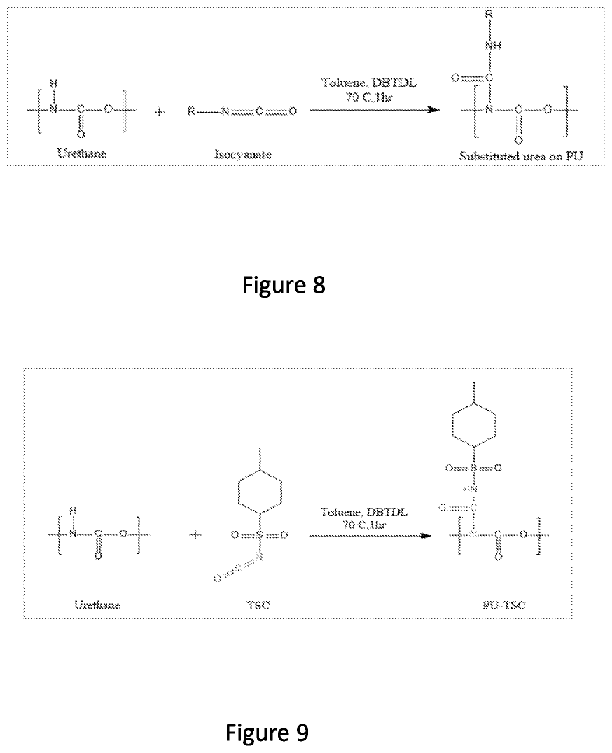

To observe a strong covalent bond between the ‘stock product’ (PEI-S-MPC, e.g., bPEI-S-MPC) and the surface of PU, a third party, diisocyanate molecule, was used. Urethane linkage, which can be found in the backbone of PU, consist of a secondary amine. The reaction between a secondary amine group and isocyanate functional group results in the formation of a substituted urea linkage, as shown in FIG. 8. This reaction occurs at 70° C., using toluene as the solvent and dibutyltin dilaurate (DBTDL) to catalyze the reaction.

Model Reaction with Toluenesulfonyl Isocyanate (TSC)

TSC molecule was used to model the reaction between isocyanate end group and the secondary amine that is found in urethane linkage. TSC consist of a sulfonyl group, which can facilitate the analysis of the product. FIG. 9 shows a scheme of a reaction between PU and TSC. The reaction was analyzed using ATR-FTIR and XPS.

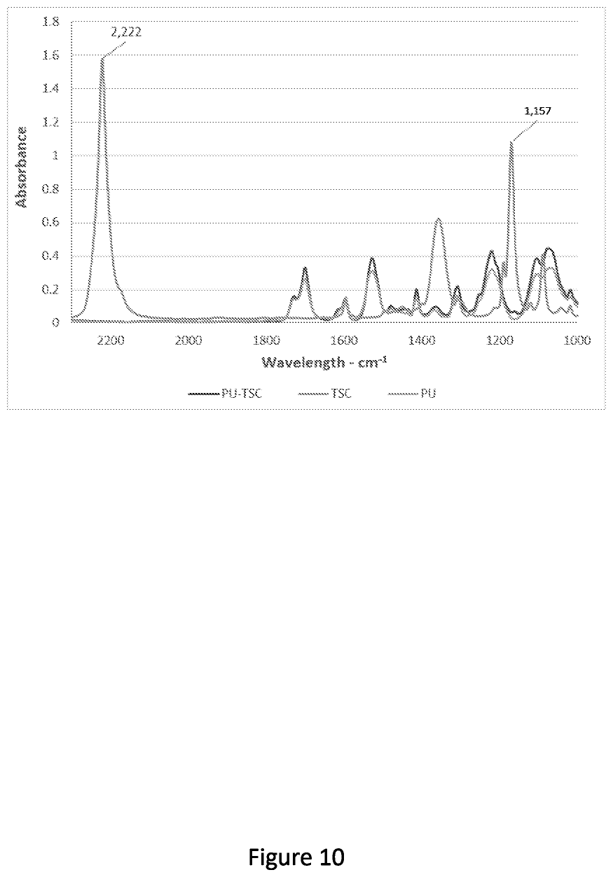

FIG. 10 shows ATR-FTIR absorption of untreated PU, TSC and the treated surface PU-TSC, according to some embodiments. The peak at 1157 cm⁻¹ stands for the absorption of the sulfonyl group. This peak could be found on the treated PU surface, indicating the presence of sulfonyl groups after the treatment. Additionally, the peak at 2222 cm⁻¹, which represents the isocyanate group could not be detected on the treated PU surface. These two findings indicate for the binding of TSC on PU surfaces. Moreover, the results of XPS analysis, which are given in Table 2, confirms the presence of sulfur atoms on the treated PU surfaces.

TABLE 2
The atomic content on the surface of
PU-TSC compared to PU:

| | Atomic content [%] | | | |
|---|---|---|---|---|
| | C | N | O | S |
| PU neat | 73.72 | 1.58 | 22.08 | 0 |
| PU-TSC | 69.88 | 4.41 | 22.91 | 1.47 |

Optimization of the Reaction Conditions

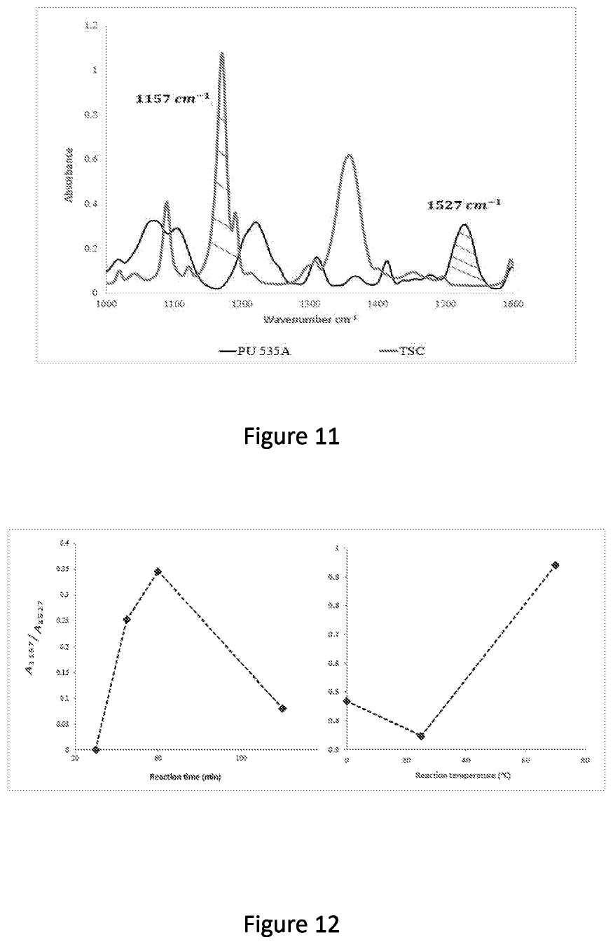

Reference is now made to FIG. 11, which shows a scheme of the peak areas that was analyzed for the optimization of the reaction of PU with isocyanate functional group, according to some embodiments. The integration of desired peaks in ATR-FTIR spectrum was used as a quantitative method to optimize the conditions of the reaction. The peak area of the sulfonyl group at 1157 cm⁻¹ (A1157) was divided by the peak area of a reference absorption peak in PU spectrum, 1527 cm−1 (A1527). The ration between the peaks was taken as the comparison factor. The parameters that were optimized were the duration and the temperature of the reaction.

Reference is now made to FIG. 12, which shows the influence of the duration (left graph) and the temperature (right graph) of the reaction on the quantity of conjugated molecules according to the integration ratio factor A1157/A1527, according to some embodiments. It was found that the ideal treated PU surface is observed in a temperature of 70° C. for 60 min.

Diisocyanates as Efficient Mediators

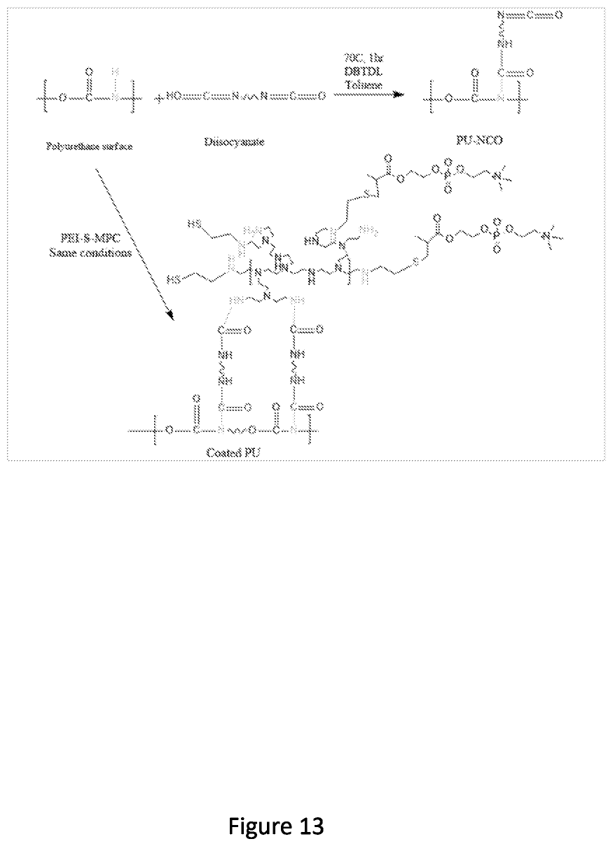

Diisocyanate may be used, in accordance with some embodiments, as coating mediator enables the simplification of the coating application on PU surfaces. As the ‘stock product’, PEI-S-MPC/bPEI-S-MPC, consists of free amine end-groups, it can bind to a free isocyanate group that can be found on PU surfaces using the same reaction as the functionalization step. The scheme of the reaction is shown in FIG. 13, which shows a scheme of the application of the ‘stock product’ (bPEI-S-MPC) on PU surface through functionalization of the PU surface, according to some embodiments. As seen in FIG. 13, the PU surface is functionalized using diisocyanate substance. One isocyanate group is covalently attached to the surface while the other isocyanate group is free and available for further reaction. Then, the covalent attachment of the ‘stock product’ to the isocyanate free groups of the treated PU takes place via free primary and/or secondary amines which present in the stock product, resulting in a urea bond.

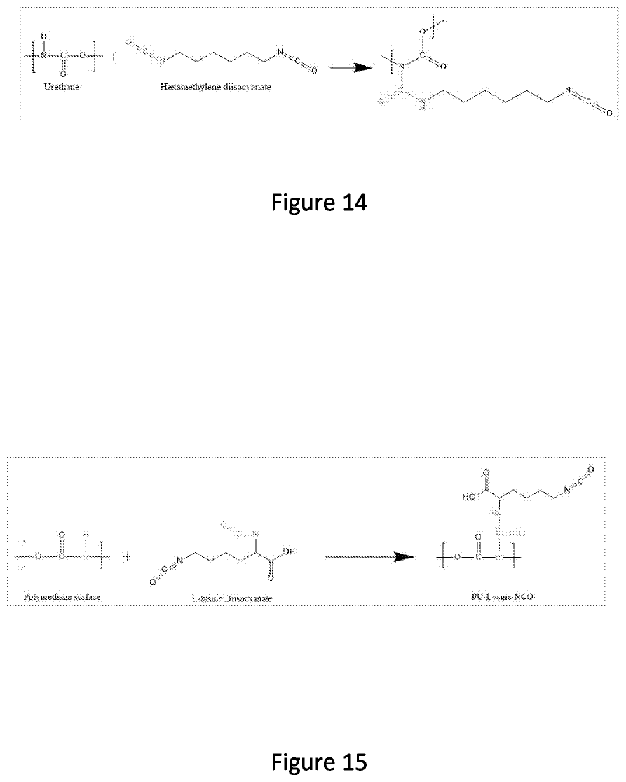

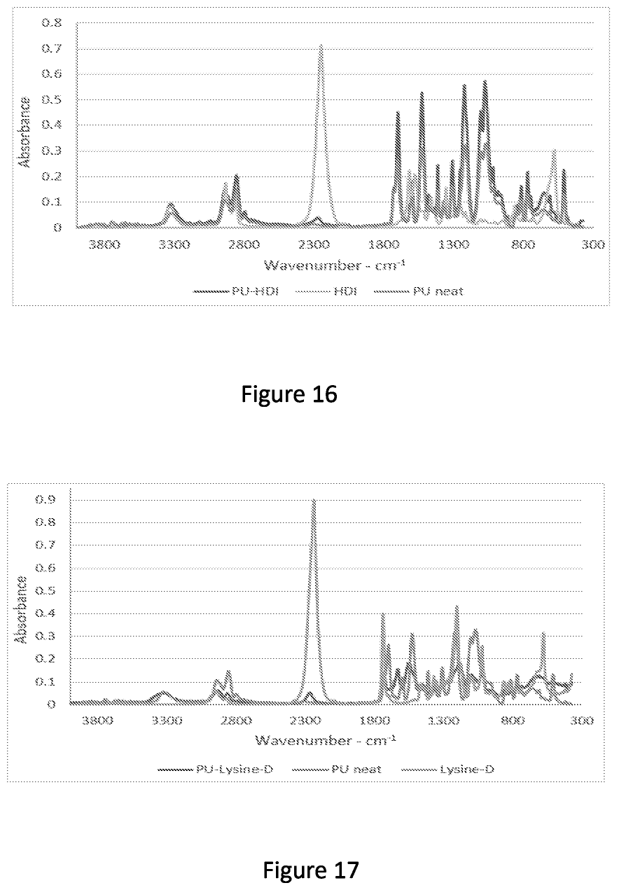

Hexamethylene diisocyanate (HDI) and L-lysine diicosyanate (lysine-D) were substituted on PU surfaces. Reference is now made to FIG. 14, which shows a scheme of a functionalization of PU surface using hexamethylene diisocyanate (HDI), according to some embodiments and to FIG. 15, which shows a scheme of a functionalization of PU surface using L-lysine diicosyanate (lysine-D), according to some embodiments. Lysine is an amino acid that is found in human proteins. The use of lysine-D can facilitate the FDA approve of the coating. The treated PU surfaces with both HDI and lysine-D were analyzed using ATR-FTIR analysis. FIG. 16 shows an ATR-FTIR spectrum of PU-HDI (produced in a process according to FIG. 14), according to some embodiments, and FIG. 17 shows an ATR-FTIR spectrum of PU-lysine-D (produced in a process according to FIG. 15), according to some embodiments.

The Application of the Coating Complex on PU Films

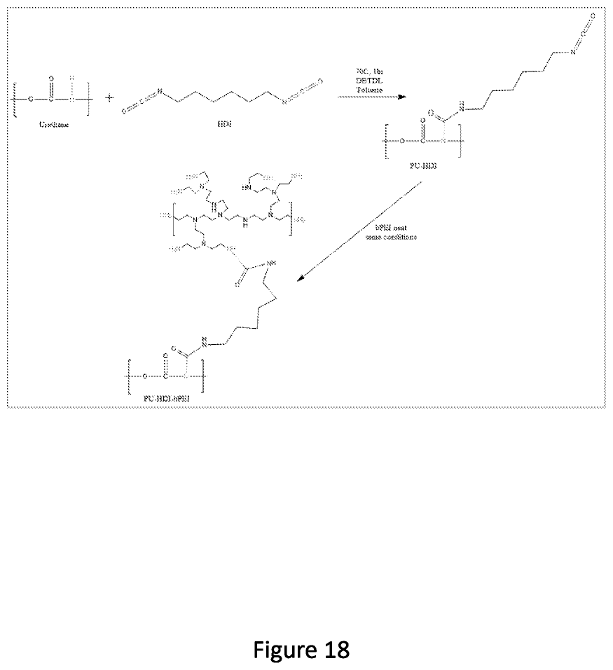

A proof of concept for the application of the coating was made through the conjugation of bPEI on functionalized PU surface (PU-HDI), to produce PU-HDI-bPEI as shown in FIG. 18, according to some embodiments.

XPS analysis was performed for PU surface, which was coated with bPEI (PU-HDI-bPEI, produced according to FIG. 18) and was compared to untreated PU surface and activated PU surface (PU-HDI). The results are listed in Table 3 below. First, it could be seen that neat PU surface consists of 24 fold Oxygen atoms compared to Nitrogen. O/N ratio decreased dramatically after surface treatment, means that the neat PU surface composition was shielded with a different layer. N/C ratio increased after the addition of isocyanate groups and the conjugation of bPEI. Nitrogen atoms content increased in 4 fold on PU-HDI-bPEI compared to a neat PU surface. This fact confirms the presence of bPEI on the surface, since bPEI is reach in Nitrogen atoms.

TABLE 3
The atomic content of PU-HDI-bPEI
as detected using XPS analysis

| | Atomic content [%] | | | | |
|---|---|---|---|---|---|
| | C | N | O | O/N | N/C |
| PU neat | 71.49 | 2.05 | 50.17 | 24.47 | 0.03 |
| PU-HDI | 69.61 | 12.07 | 15.64 | 1.3 | 0.17 |
| PU-HDI- | 71.29 | 71.29 | 18.28 | 2.1 | 0.12 |
| bPEI | | | | | |

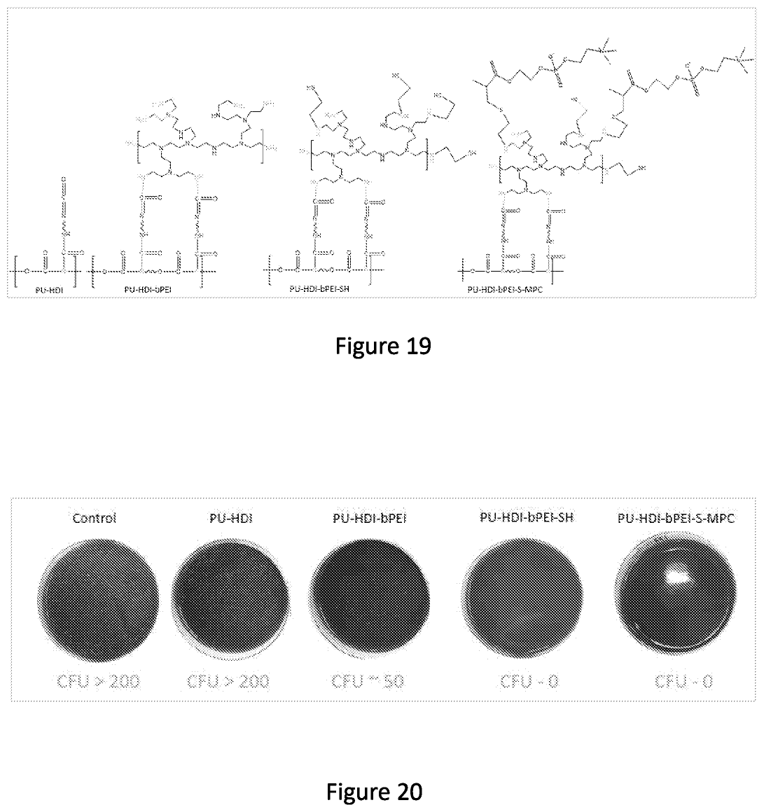

A series of samples were made to characterize the PU coating that have been developed. FIG. 19 shows a scheme of this sample series, according to some embodiments. All of the samples were coated using HDI as the surface diisocyanate activator. As a control, a sample of neat PU surface was exposed to the procedure conditions without the reactants, i.e. the solvent, temperature, initiator and duration of the procedure.

Antimicrobial Test

Reference is now made to FIG. 20, which shows blood-agar petri dishes containing the sample series shown in FIG. 19, which went through JIS Z2801:200 test for antimicrobial properties. As a control, a sample of neat PU surface was exposed to the procedure conditions without the reactants, i.e. The solvent, temperature, initiator and duration of the procedure.

It can be seen that PU-HDI-bPEI-SH and PU-HDI-bPEI-S-MPC show good antimicrobial results—colony-forming unit (CFU)—0, compared to the control (CFU>200), PU-HDI (CFU>200) and compared to PU-HDI-bPEI (CFU˜50).

Coefficient of Friction (COF)

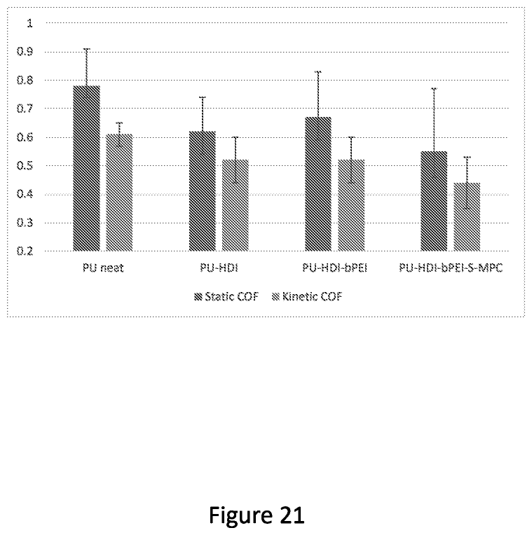

Reference is now made to FIG. 21, which shows COF of the sample series shown in FIG. 19, on PMMA in the presence of PBS. It can be seen that PU-HDI-bPEI-S-MPC shows improved lubrication properties compared to the other samples.

Hemolysis Ratio

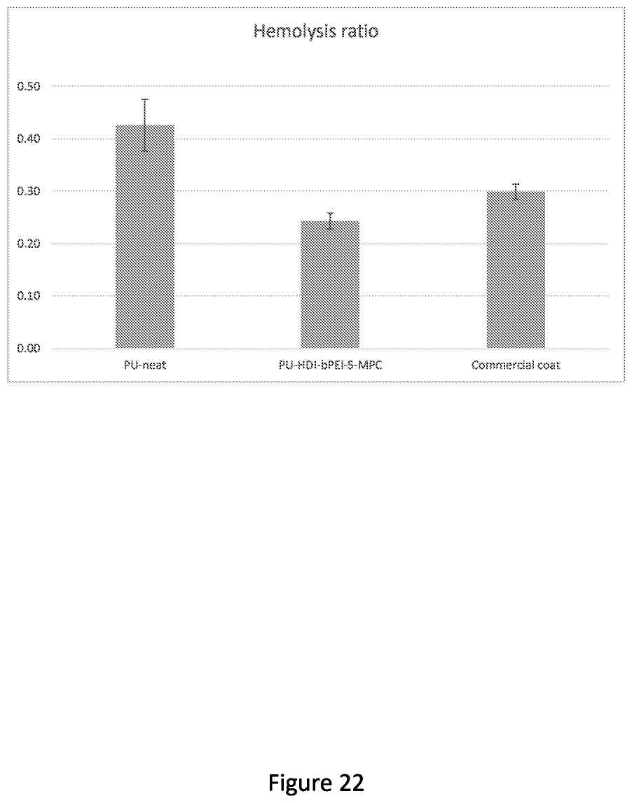

Reference is now made to FIG. 22, which shows hemolysis ratio of PU-HDI-bPEI-S-MPC compared to a hydrogel coat which can be found in medical use today. As a control, hemolysis ratio was calculated for neat PU surface, according to some embodiments.

It can be seen that PU-HDI-bPEI-S-MPC shows improved (lower) hemolysis ratio compared to the other samples.

Direct Thiolation of Polyurethane Using Ethylene Sulfide (ES)

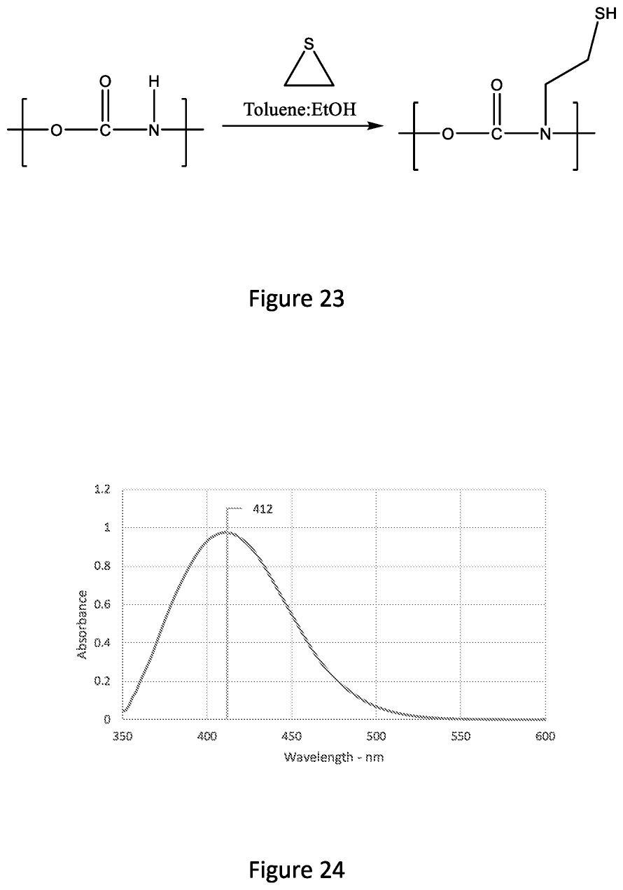

As disclosed hereinabove, there is provided herein, in accordance with additional or alternative embodiments, a method for the conjugation of a therapeutic/antithrombogenic compound (such as MPC) onto PU surfaces. FIG. 23, shows a scheme of direct thiolation of PU surface, utilizing ES, according to some embodiments. This approach may be used for direct conjugation via thiol-based reactions, such as thiol-ene with allyl or methacrylate bearing molecules (such as MPC), isocyanate bearing molecules, epoxides and the like.

Experimental

PU surface was soaked in a solvent mixture of toluene and ethanol (9:1, respectively). After 10 min of reflux under nitrogen atmosphere, 500 μl of ES were added dropwise over 90 sec. the reaction was refluxed for another 1.5 hours following three washing steps in excess of toluene for 15 min to remove all unreacted ES molecules. The modified PU surface was analyzed using UV-VIS spectrophotometer, elemental analysis and immunofluorescence. Similar experiments are performed using other toluene and ethanol ratios, such as 8:2, respectively and in toluene 100%, with addition of 100 μl-2 ml of ES, e.g., dropwise over 10-120 sec. The reaction is refluxed for 10 min-2 hours.

UV-VIS Analysis

Ellman's reagent was used to detect free thiol groups on treated (thiolated) PU surface. FIG. 24 shows the UV-VIS absorbance curve of Ellman's reagent decomposition products after the exposure to thiolated PU surface (PU-SH), according to some embodiments. Following the standard protocol for using the Ellman's reagent, the absorbance at a wavelength of 412 nm approve the presence of free thiol groups on the PU-SH surface.

Elemental Analysis

Table 2 shows the atomic percent distribution that was obtained from elemental analysis. After the thiolation of PU surface, 0.73% of the detected atoms was found to be sulfur. Visually, there was no significant change between PU and PU-SH.

TABLE 2
The atomic content of PU and PU-SH as
was observed by elemental analysis:
Atomic content [%]

| | PU Neat | PUSH |
|---|---|---|
| C | 67.16 | 65.64 |
| H | 8.41 | 8.43 |
| N | 4.19 | 5.28 |
| S | 0.1 | 0.73 |
| O | 19.33 | 19.14 |

Fluorescent Probe

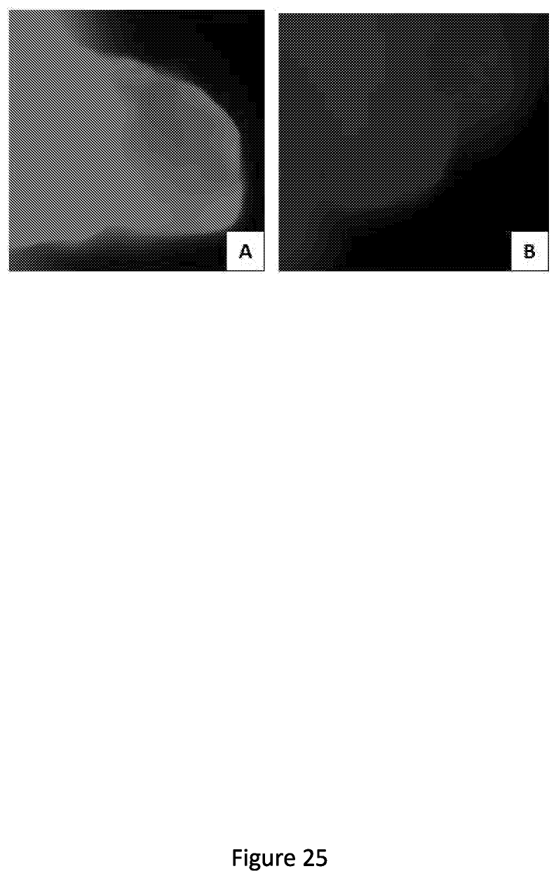

Fluorescein-O-methacrylate was used as a fluorescent probe for reactive thiol groups. Thiol-ene click reaction was performed to conjugate reactive thiols with methacrylate groups. The reaction occurred in methanol using DMPA as the initiator and the observed product is called PU-S-Fluorescein. As a reference, the same procedure took place without the addition of DMPA. FIG. 25 shows fluorescent microscopy of fluorescein-O-methacrylate grafted PU-SH (A) compared to a reference (B), according to some embodiments. A significantly strong fluorescent signal was obtained from the fluorescein-O-methacrylate grafted PU-SH, compared to the reference. the fluorescent signal assures the presence of fluorescein group on the surface of the fluorescein-O-methacrylate grafted PU-SH sample. The reference sample was not fluorescent. These finings eliminate the possibility of physical absorption of the fluorescein-O-methacrylate, proving the covalent bond of fluorescein-O-methacrylate to the thiolated PU surface.

Ex-Vivo Thrombogenic Protocol

Samples (Nitinol discs, 5 mm in diameter, coated or uncoated) are being placed on the bottom of a 50 ml PTFE flask. 8 ml of fresh blood from healthy pigs is casted onto the samples and the flasks are gently shaken at 50 rpm for 3 hours.

Fixation of the samples occurs in 4% formaldehyde in PBS, following by dehydration of the samples using washing with elevated concentration of ethanol and the surfaces are being examined using scanning electron microscopy (SEM).

In Vivo Thrombogenic Evaluation

Through the right femoral artery of healthy rabbits, the samples (Nitinol wires, coated or uncoated) are administered to abdominal aorta for 3 hours.

The segment of abdominal aorta is then removed from the animal, its content is emptied into a petri dish containing 50 ml of a 0.9% saline solution, and the contents of the dish is photographed and examined for the presence of clots on the device.

The morbidity and mortality of the animals is examined daily. The thrombogenic potential is evaluated using SEM as follows:

0—no clot

1—Few macroscopic standards of fibrin

2—Several small thrombi

3—Two or more large thrombi

4—A single thrombus forming a cast of the isolated segment.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### REFERENCES

[1] G. D. I. Francolini, “Efficacy of Antiadhesive, Antibiotic and Antiseptic Coatings in Preventing Catheter-Related Infections: Review,” J. Chemother., vol. 13, pp. 595-606, 2001.
[2] J. Simon, “What's next for hydrophilic coatings?,” Medical Design, 2009.
[3] F. P. Documents, “LUBRICIOUS COATINGS FOR MEDICAL DEVICES,” 2002.
[4] A. I. Alomari and A. Falk, “The Natural History of Tunneled Hemodialysis Catheters Removed or Exchanged: A Single-Institution Experience,” SIR, vol. 9, pp. 227-235, 2004.
[5] F. P. Documents, “HYDROPHILIC LUBRICITY COATING FOR MEDICAL DEVICES COMPRISING A HYDROPHOBIC TOP COAT,” 2001.
[6] Y. Chen, A. Dai, Y. Shi, Z. Liu, M. Gong, and X. Yin, “Effectiveness of silver-impregnated central venous catheters for preventing catheter-related blood stream infections: a meta-analysis,” Int. J. Infect. Dis., vol. 29, pp. 279-286, 2014.
[7] K. D. Mandakhalikar, R. R. Chua, and P. A. Tambyah, “New Technologies for Prevention of Catheter Associated Urinary Tract Infection,” Springer Sci., 2016.
[8] C. J. Van Rooden, E. F. Schippers, R. M. Y. Barge, F. R. Rosendaal, and H. F. L. Guiot, “Infectious Complications of Central Venous Catheters Increase the Risk of Catheter-Related Thrombosis in Hematology Patients: A Prospective Study,” vol. 23, no. 12, pp. 2655-2660, 2005.
[9] FDA, “Intravascular Medical Devices—FDA Safety Communication—Lubricious Coating Separation,” U.S. Food and Drug Administration, 2015.
[10] J. Simon, “The Crucial Divide between Hydrophobic and Hydrophilic Coatings,” Med-Tech Innovation, 2011.
[11] S. Michael J. Micklus, both of David T. Ou-Yang, Raritan, and NJ., “COATED SUBSTRATE HAVING A LOW COEFFICIENT OF FRICTION HYDROPHILIC COATING AND A METHOD OF MAKING THE SAME,” 1978.
[12] M. R. M. R. Stephe and B. wright Mlramar, “CATHETER AND HYDROPHILIC, FRICTION-REDUCING COATING THEREON,” 1991.
[13] K. a. Kaźmierska, K. Kuc, and T. Ciach, “Polyvinylpyrrolidone-polyurethane interpolymer hydrogel coating as a local drug delivery system,” Acta Pol. Pharm.—Drug Res., vol. 65, no. 6, pp. 763-766, 2008.
[14] M. Kenward and G. W. Slater, “Combinatorial design of passive drug delivery platforms,” Int. J. Pharm., vol. 339, pp. 91-102, 2007.
[15] Haslam John H, “Method of coating surface with transparent film and product resulting therefrom,” 1956.
[16] M. Rooijmans, “HYDROPHILIC COATING,” 2013.
[17] J. M. R. R. Erich Bauman, Peter Hagmann, John Dallas Pruitt, “Silicone hydrogel lenses with nano-textured surfaces,” 2016.
[18] A. L. Lewis, “Phosphorylcholine-based polymers and their use in the prevention of biofouling,” Elsevier Sci., vol. 18, pp. 261-275, 2000.
[19] “Membrane asymmetry and blood coagulation,” Nature, vol. 268, pp. 358-360, 1977.
[20] “What is choline?,” Choline info.org.
[21] S. Chen, J. Zheng, L. Li, and S. Jiang, “Strong Resistance of Phosphorylcholine Self-Assembled Monolayers to Protein Adsorption: Insights into Nonfouling Properties of Zwitterionic Materials,” Am. Chem. Soc., no. 17, pp. 14473-14478, 2005.
[22] M. Sin, S. Chen, and Y. Chang, “Hemocompatibility of zwitterionic interfaces and membranes,” Polym. J., vol. 46, no. 8, pp. 436-443, 2014.
[23] “Phosphorylcholine,” Pubchem.
[24] S. F. Long, S. Clarke, M. C. Davies, A. L. Lewis, G. W. Hanlon, and A. W. Lloyd, “Controlled biological response on blends of a phosphorylcholine-based copolymer with poly (butyl methacrylate),” vol. 24, pp. 4115-4121, 2003.
[25] M. Jawanda, B. F. L. Lai, and J. N. Kizhakkedathu, “Linear and hyperbranched phosphorylcholine based homopolymers for blood biocompatibility,” Polym. Chem., pp. 3140-3146, 2013.
[26] S. D. Pacetti, “Implantable medical devices with a topcoat layer of phosphoryl choline acrylate polymer for reduced thrombosis, and improved mechanical properties,” 2009.
[27] N. D. Thierry Glauser, Stephen D. Pacetti, Syed F. A. Hossainy, “Phosphoryl choline coating compositions,” 2005.
[28] D. Pecosky and A. Park, “One-step phosphorylcholine-linked polymer coating and drug loadin of stents,” 2014.
[29] L. Aravindan, K. A. Bicknell, G. Brooks, V. V Khutoryanskiy, and A. C. Williams, “A Comparison of Thiolated and Disulfide-Crosslinked Polyethylenimine for Nonviral Gene Delivery,” pp. 1163-1173, 2013.
[30] ES 2628210T3-Polyurethane having an antithrombogenic coating.

1.-25. (canceled)

26. A method of coating a polyurethane surface of an insertable medical device, the method comprising:
obtaining an insertable medical device or a part thereof comprising a polyurethane surface;
performing a direct thiolization of the polyurethane surface to produce thiolated polyurethane surface comprising free thiol groups, the direct thiolization comprises a direct reaction between a secondary amine of the polyurethane surface and ethylene sulphide (ES) to form a covalent bond between the amine and the free thiol group;
reacting the thiolated polyurethane surface with a therapeutic/antithrombogenic compound having a vinyl/methacrylate functional group through thiol-ene click reaction, to produce an insertable medical device coated with a therapeutic/antithrombogenic and abrasion (delamination)-resistant coating.

27. The method of claim 26, wherein the direct thiolization of the polyurethane surface to produce thiolated polyurethane surface is devoid of a pre-treatment of the polyurethane surface.

28. The method of claim 26, wherein the direct thiolization of the polyurethane surface to produce thiolated polyurethane surface is devoid of plasma pre-treatment, chemical pre-treatment, flame pre-treatment, corona pre-treatment or any combination thereof, of the polyurethane surface.

29. The method of claim 26, wherein the therapeutic/antithrombogenic compound having a vinyl/methacrylate functional group comprises zwitterionic methacrylate.

30. The method of claim 26, wherein the zwitterionic methacrylate comprises sulfobetaine methacrylate, phosphorylcholine methacrylate or a combination thereof.

31. The method of claim 29, wherein the zwitterionic methacrylate comprises 2-methacryloyloxylethyl phosphorylcholine (MPC) and wherein the coated polyurethane surface is (PU-S-MPC).

32. The method of claim 26, wherein the therapeutic/antithrombogenic compound having a vinyl/methacrylate functional group comprises Linalool, Limonene, Citral or any combination thereof.

33. An insertable medical device having a polyurethane surface coated according to the method of claim 26.

34. A method for preparing a stock product for use as a coating material for coating a polyurethane surface of an insertable medical device, the method comprising:
obtaining a thiolated polyethyleneimine (PEI-SH); and
reacting the thiolated polyethyleneimine (PEI-SH) with a therapeutic/antithrombogenic compound having a vinyl/methacrylate functional group through thiol-ene click reaction to produce a stock product comprising polyethyleneimine-thiol-therapeutic/antithrombogenic compound conjugate having free primary and/or secondary amines capable of binding to an activated surface of the insertable medical device.

35. The method of claim 34, wherein the therapeutic/antithrombogenic compound having a vinyl/methacrylate functional groups comprises zwitterionic methacrylate.

36. The method of claim 35, wherein the zwitterionic methacrylate comprises sulfobetaine methacrylate, phosphorylcholine methacrylate or a combination thereof.

37. The method of claim 35, wherein the zwitterionic methacrylate comprises 2-methacryloyloxylethyl phosphorylcholine (MPC) and wherein the stock product comprises polyethyleneimine-thiol-MPC (PEI-S-MPC) conjugate.

38. The method of claim 34, wherein the therapeutic/antithrombogenic compound having a vinyl/methacrylate functional groups comprises Linalool, Limonene, Citral or any combination thereof.

39. The method of claim 34, wherein obtaining the thiolated-polyethyleneimine (PEI-SH) comprising reacting polyethyleneimine (PEI) with ethylene sulphide (ES), halogen-alkyi thiol, cysteine, bromopyridine thiol, bromobenzoxazole thiol, chloropyridine thiol, halobenzo thiazole thiol, chloropyrimidine thiol, halo-phenyl thiazole thiol or any combination thereof.

40. The method of claim 34, wherein the polyethyleneimine (PEI) and/or the thiolated polyethyleneimine (PEI-SH) comprises brunched polyethyleneimine (bPEI) and/or thiolated-brunched polyethyleneimine (bPEI-SH), respectively.

41. A method for preparing polyethyleneimine-thiol-2-methacryloyloxylethyl phosphorylcholine (PEI-S-MPC) for use as a stock product for coating a surface of an insertable medical device, the method comprising:
obtaining a thiolated polyethyleneimine (PEI-SH); and
reacting the thiolated polyethyleneimine (PEI-SH) with 2-methacryloyloxylethyl phosphorylcholine (MPC) through thiol-ene click reaction to produce brunched polyethyleneimine-thiol-2-methacryloyloxylethyl phosphorylcholine (PEI-S-MPC) having free primary and/or secondary amines capable of binding to an activated surface of the insertable medical device.

42. The method of claim 41, wherein obtaining the thiolated-polyethyleneimine (PEI-SH) comprising reacting polyethyleneimine (PEI) with ethylene sulphide (ES), halogen-alkyi thiol, cysteine, bromopyridine thiol, bromobenzoxazole thiol, chloropyridine thiol, halobenzo thiazole thiol, chloropyrimidine thiol, halo-phenyl thiazole thiol or any combination thereof.

43. A stock product for use in coating an activated polyurethane surface of an insertable medical device, the stock product prepared according to claim 34.

44. A method of coating a polyurethane surface of an insertable medical device, the method comprising:
obtaining an insertable medical device or a part thereof comprising a functionalized polyurethane surface having free isocyanate groups;
reacting the functionalized polyurethane surface with the stock product prepared according to the method of claim 34, the stock product comprising a conjugate of polyethyleneimine-thiol-therapeutic/antithrombogenic compound having free primary and/or secondary amines capable of binding to the free isocyanate groups of the polyurethane surface.

45. The method of claim 44, wherein the polyurethane surface is functionalized using diisocyanate substance to produce.
